(19) United States
(12) Patent Application Publication
(10) Pub. No.: US 2021/0238536 A1
(43) Pub. Date: Aug. 5, 2021

(54) CONTAINER FOR SEPARATING MICROCARRIERS FROM CELL CULTURE FLUIDS

(71) Applicant: EMD Millipore Corporation, Burlington, MA (US)

(72) Inventor: Martin Morrissey, Billerica, MA (US)

(21) Appl. No.: 16/330,413

(22) PCT Filed: Oct. 23, 2017
(86) PCT No.: PCT/US2017/057856

§ 371 Date: Mar. 5, 2019

Related U.S. Application Data
(60) Provisional application No. 62/416,309, filed on Nov. 2, 2016.

(51) Int. Cl. C12M 1/00; C12M 1/12; C12M 1/26

(52) U.S. Cl. CPC C12M 47/02; C12M 23/14; C12M 33/14; C12M 25/16; C12M 25/14

(57) ABSTRACT

Containers for separating microcarriers from a cell culture fluid that offer a greater efficiency of filtration of cell culture fluids containing microcarriers relative to systems described in the art. The container may include a first compartment that may include a sterile collapsible bag, an inlet port providing a fluid path into the first compartment and an outlet port providing a fluid path exiting the first compartment; and a second compartment fluidly connected with the inlet port of the first compartment and including a plurality of independent or discrete microcarrier receiving regions defined by boundary walls which are partially or fully porous and having a porosity sufficient to retain the microcarriers inside the second compartment, while allowing the cell culture fluid to pass through the second compartment into the outlet port of the first compartment, where the cell culture fluid can be collected.

This application claims priority of U.S. Provisional Application Ser. No. 62/416,309 filed Nov. 2, 2016, the disclosure of which is hereby incorporated by reference.

### BACKGROUND

Microcarriers are typically used for the culturing of adherent or anchorage-dependent cells and are widely used in the pharmaceutical industry for the same. Microcarriers may be used for culturing adherent cells which are used for manufacturing of certain biologics or vaccines, or for culturing certain types of cells (e.g., stem cells), where the stem cells themselves are the intended product.

Microcarriers typically harbor surface characteristics or chemistries which enable or facilitate the attachment of cells onto the microcarriers. Bioreactors are used for culturing of adherent cells involving microcarriers. Once the cells reach a certain density or the cell culture process is completed, the cell culture fluid needs to be separated from the microcarriers for further processing of either the cell culture fluid itself (e.g., in case of a secreted therapeutic protein, e.g., a monoclonal antibody) or the microcarriers with cells attached thereto (e.g., in case of stem cells). Further, it is often desirable to separate the microcarriers from the cell culture fluid so that the microcarriers may be re-used following sterilization.

In general, several methods have been described to remove the cells from the microcarriers, e.g., by treatment of microcarriers with trypsin, EDTA or similar agents to release the cells from the microcarriers. Several processes have also been described for separating microcarriers from cell culture fluid. For example, for bulk processing of large volumes, the traditional method of separation has been to let the microcarriers settle, e.g., on a tilted settling table of stacked surfaces or in a shallow container. Once the cells have settled, it is possible to harvest most of the supernatant by decanting and recovery of product can be enhanced by repeating the settling step. However, the required time for such a process can be too long for efficient recovery and product can deteriorate.

Alternatively, filters have been described to separate microcarriers from cell culture solutions. For example, one conventional system includes a filtration screen incorporated into a disposable receiving bag, whereby the solution containing the microcarriers is transferred into the receiving bag via a circuit feeding into the receiving bag through a fitment that transects the receiving bag wall. An inlet fitment which transfers the microcarrier suspension across the wall of a flexible receiving bag is divided into two chambers by means of a planar mesh sheet, such that the first chamber fed by the inlet fitment is where the microcarriers accumulate and the second chamber receives the liquid solution free of microcarriers.

Another conventional system includes a filter assembly for separating microcarriers from a fluid medium, which includes a collapsible container around a sterile compartment adapted to hold a fluid; an inlet port through which fluid flows into the compartment; an outlet port through which fluid flows out of the compartment; and a filter disposed within the compartment, which divides the compartment into an inlet chamber that is fluidly coupled with the inlet port and an outlet chamber that is fluidly coupled with the outlet port, and which allows a medium to pass through the filter while preventing microcarriers to pass through.

Separation of the microcarriers from the cultured solution that includes the detached cells may be achieved by passing the solution through a rigid container having a horizontal screen that extends across the rigid container. The screen is a rigid mesh that allows the cultured fluid to pass through but prevents the microcarriers from doing so. However, as the microcarriers build up on the screen, they begin to clog the screen and prevent the fluid from passing therethrough. Once the screen is clogged, the process stops until the screen is unclogged. Furthermore, once the process is completed, the rigid container and related screen must be cleaned and sterilized before it can be re-used. These process steps can be expensive and time consuming.

Anchorage dependent cells have a tendency or requirement to “spread” on substrates and thus occupy relatively large surface areas relative to cell numbers. This greatly complicates processes for production of anchorage dependent cell products. By example, a 75 cm² culture surface may yield an essentially negligible 1×10⁵⁻⁶ cells, a few micrograms of total wet cell weight, and far less than that of any useful pharmaceutical product. Thus, despite years of attempting to overcome limitations of planar surface attachment, it has been highly impractical to grow anchorage dependent cells on flat surfaces for production.

Accordingly, what is needed in the art are methods and/or systems that can alleviate one or more of the above problems.

### SUMMARY

Embodiments described herein relate to containers for separating microcarriers from a cell culture fluid. The containers described herein offer a greater efficiency of filtration of cell culture fluids containing microcarriers relative to systems described in the art. For example, in case of filtration systems of the prior art, e.g., the ones described above, once the bag fills with microcarriers, a smaller and smaller percentage of the surface area of the microcarriers is in contact with the filtration vessel (e.g., bag or pouch), thereby slowing down or impeding the filtration process and decreasing the overall filtration efficiency. The containers described herein have a high surface area, resulting in an increase in the efficiency of filtration.

In some embodiments, a container for separating microcarriers from a cell culture fluid is provided, the container comprising a first compartment that may include a sterile collapsible bag, an inlet port providing a fluid path into the first compartment and an outlet port providing a fluid path exiting the first compartment; and a fully enclosed second compartment fluidly connected with the inlet port of the first compartment and including boundary walls which are partially or fully porous and having a porosity sufficient to retain the microcarriers inside the second compartment, while allowing the cell culture fluid to pass through the second compartment into the outlet port of the first compartment, where the cell culture fluid can be collected.

In certain embodiments, the fully enclosed second compartment has a plurality of boundary walls defining a plurality of independent or discrete microcarrier receiving regions. The regions are independent or discrete in that microcarriers in one independent or discrete region do not directly interact with, and are not in contact with, microcarriers in another independent region. In some embodiments, each of the microcarrier receiving regions is a pouch.

In certain embodiments, there are a plurality of fully enclosed compartments, each fluidly connected with the inlet port of the first compartment and including boundary walls which are partially or fully porous and having a porosity sufficient to retain the microcarriers inside the second compartment, while allowing the cell culture fluid to pass through the second compartment into the outlet port of the first compartment, where the cell culture fluid can be collected.

In some embodiments, a method for separating microcarriers from a cell culture fluid is provided, the method comprising:

providing a cell culture fluid including microcarriers;

providing a container including a first compartment and a fully enclosed second compartment disposed inside the first compartment, where the first compartment includes a sterile collapsible bag, an inlet port providing a fluid path into the first compartment, and an outlet port providing a fluid path exiting the first compartment; and the second compartment is fluidly connected with the inlet port of the first compartment and includes boundary walls which are partially or fully porous and have a porosity to retain microcarriers inside the second compartment, while allowing fluid to pass through the second compartment into the outlet port; the boundary walls defining independent or discrete microcarrier receiving regions; and

flowing the cell culture fluid including microcarriers through the inlet port of the first compartment, such that microcarriers flow into the fully enclosed second compartment where they are trapped and accumulate inside the second compartment, and the remaining cell culture fluid flows out of the second compartment through the outlet port of the first container,

thereby separating the microcarriers from the cell culture fluid.

In some embodiments, the second compartment comprises a plurality of independent or discrete microcarrier receiving regions, each comprising a top portion providing a fluid path for cell culture fluid containing microcarriers to enter the microcarrier receiving region, side walls, and a bottom portion that is sufficiently porous to allow the cell culture fluid to pass while retaining the microcarriers in the microcarrier receiving region.

In some embodiments, the plurality of microcarrier receiving regions of the second compartment are connected to a plenum to form a manifold. The plenum may be comprised of a rigid material, such as, for example, polysulphone, acrylic or polycarbonate polymers. Alternatively, it may be comprised of a flexible material such as, for example, vinyl or polyvinylchloride polymers. In some embodiments, the plenum distributes the cell culture fluid containing microcarriers to each of the plurality of microcarrier receiving regions.

### BRIEF DESCRIPTION OF THE DRAWINGS

FIG. 1 is a schematic diagram of a container for separating microcarriers in accordance with certain embodiments;

FIG. 2 is a is schematic diagram of a container for separating microcarriers in accordance with another embodiment;

FIG. 3 is a schematic diagram of a container for separating microcarriers in accordance with certain embodiments; and

FIG. 4 is another schematic diagram of container for separating microcarriers in accordance with certain embodiments.

### DETAILED DESCRIPTION

A more complete understanding of the components, processes and devices disclosed herein can be obtained by reference to the accompanying drawings. The figures are merely schematic representations based on convenience and the ease of demonstrating the present disclosure, and is, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

Anchorage-dependent cells, including many genetically modified animal cells, attach to surfaces by processes that include electrostatic/hydrophobic interactions, production of self-attachment matrices or attachment to coatings of polyamino acids (e.g. polylysine) or a variety of “scaffolding” proteins including collagens, laminins, fibronectins and other “RGD” peptides. These mimic cell attachment substrates that secure cells in natural environments, Anchorage-dependence is an essential requirement because the attachment process itself provides signals into the cells that control genetic and synthetic processes and specifically the production of desired products.

Batch Methods

Batch mode microcarrier cell culture simply involves providing a combination of cell coated microcarriers and nutrient medium in a container in a manner supportive to cellular health: gases, buffers, anabolic carbon sources and growth factors are provided and optimized for maximum production of the desired product. Once the optimized concentration of product is reached, the suspension is separated from the microcarriers in some way and then subjected to downstream processing.

A fed-batch mode is similar to the batch mode in that products are removed only at the end of the run, but differs in that nutrients are added at multiple intervals during the process, with the object of improving the recovery of product.

Perfusion (Continuous Flow) Mode

In perfusion mode, a continuous flow of fresh nutrient medium passes through the suspension of microbeads. Since microcarriers are selected to be slightly denser than the density of the medium, which is typically perfusing very slowly through the culture vessel. Thus the microcarrier weight offsets the flow vector (the “lift” factor of the moving medium) that would otherwise expel the microcarriers from the culture vessel. If the desired product is excreted into the nutrient medium, this is recovered from the effluent stream. If the product is still associated with cells attached to the microcarrier beads, or contained in the cells after they are stripped from the microcarriers by chemical or enzymatic means ((typically trypsin or “EDTA” (ethylene diamine tetraacetic acid)), then separation of the cells from the microcarriers is necessary before further processing occurs.

Thus, in the continuous or perfusion mode, the product is harvested throughout the culture period. In the Batch and Fed-Batch mode, products are removed only at the end of the process run.

Processing of Microcarrier Based Cultures

Both of these current methods will provide good capture of the microcarriers, given that the dimensions of the mesh filtration media is large relative to the concentration of the microcarriers. However, as the microcarrier capture chamber begins to fill with the microcarriers, a portion of the mesh is occluded, so the efficiency of filtration drops and processing of the fluid stream must necessarily decrease. Thus, there remains an on-going need for an apparatus and method that provides a faster, more efficient means for separating microcarrier beads or cells from the culture medium, and for recovering the microcarrier beads or cells at the time of harvest. The need for such an apparatus and method for use in the continuous or perfusion mode of cell culturing wherein nutrients are continuously added to the system and product is harvested throughout the culture period, is particularly obvious.

To overcome the exhaustion of filter medium in the capture devices heretofore known it is necessary to increase the available surface area of the capture media. The embodiments disclosed herein substantially increase the surface area of the filtration media without increasing the volume of the overall device when deployed in a receiving bag.

Embodiments disclosed herein provide devices and methods that filter microcarriers or other aggregates from cell culture solutions or process solutions in a particularly effective way, so that the filtrate of microcarrier suspension medium is efficiently separated from the microcarriers themselves. The design of the devices greatly reduces filter clogging and flow blockage expected from devices already known in the art, while at the same time providing all the advantages expected by applying similar devices in any type of sterile disposable or reusable sterilizable bioreactor. More specifically, embodiments disclosed herein relate to an improved disposable filtration device for cell microcarriers and to incorporation of the filter units into process circuits for the recovery of cells and cell products from microcarrier cell cultures. In general, the disposable filtration device and filtrate recovery devices can comprise non-porous disposable bags of any size. One embodiment is referred to as “pillow” bags and comprises two or more sheets of polymer or laminated polymer disposed facing each other and sealed or adhered together along the periphery. Alternatively, there are disposable 3-dimensional disposable bags, that is, bags that are fabricated to have three, four, five or more walls of flexible unitary or laminated nonporous polymeric material.

The objective of certain embodiments is to increase the efficiency of filtration. In certain embodiments, the surface area of the porous filtration compartment is increased by increasing the number of walls of the compartment to create a plurality of independent or discrete microcarrier receiving regions. The effective density of the bed of microcarriers that accumulate in the microcarrier regions is reduced without reducing the actual number of microcarriers used. Accordingly, for the same number of microcarriers, more microcarrier surface area is exposed to the sample or cell culture solution.

In some embodiments, a manifold or plenum may be used to direct process fluid into the second compartment or compartments.

In certain embodiments, the first compartment of the device may be a bag. The bag may carry a variable number of fitments, such as sterile ports, tubing connections and arrangements of tubing circuits. In one embodiment the bag is nonporous and comprises a flexible polyethylene material or film, and may have fitments attached to it. The term “fitment” as used herein refers to a separate object that is welded, e.g., heat welded to the nonporous bag film in order to attach it. As such, a fitment often comprises a polymeric material which can be the same or similar to the polymeric material comprising the wall of the nonporous bag. A fitment is often a more dense material than the wall of the nonporous bag, and may be added to the bag to enable a functionality. A non-limiting example of a fitment is one that forms a port. In certain embodiments, a port as described below is added to the wall of the nonporous bag in order to withdraw cell culture medium or other fluid from the interior of the nonporous bag. Such bags may be used while contained in metal tanks or bins to relieve stresses from large fluid loads.

In certain embodiments, a second compartment is contained within the first compartment, which collects filtrate from the second compartment filters. The second compartment (the filter) may be sealed to the wall of the first compartment along the top edge of the compartment such as by adhesive or heat sealing, for example. The second compartment includes a plurality of independent or discrete microcarrier receiving regions.

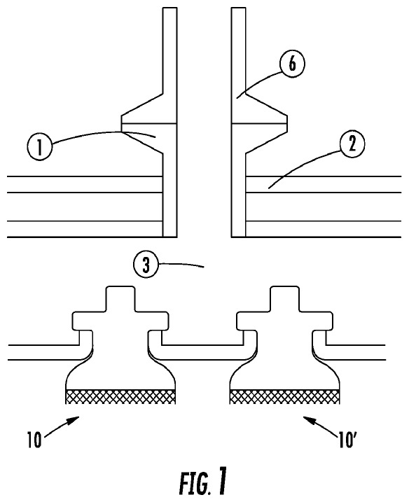

Turning now to FIG. 1, there is shown a fitment 1 that couples to an external feed tube from an external source of fluid and beads (not shown), and provides a path through into a container and into a plenum 3 in fluid communication with a plurality of independent or discrete microcarrier receiving regions 10, 10′ (partially shown). In the embodiment shown, there are two such microcarrier receiving regions 10, 10′, each of which is a mesh filtration bag. Each of the microcarrier receiving regions 10, 10′ is configured to house in its internal volume a plurality of microcarriers independently from the other; the plurality of microcarriers in the compartment 10 are independent and distinct from the plurality of microcarriers in the compartment 10′. In some embodiments, the plurality of microcarriers in each microcarrier receiving region 10, 10′ are trapped and accumulate to form a bed of microcarriers. Each microcarrier receiving region 10, 10′ may be identical (e.g., identical volumes and configuration) but need not be.

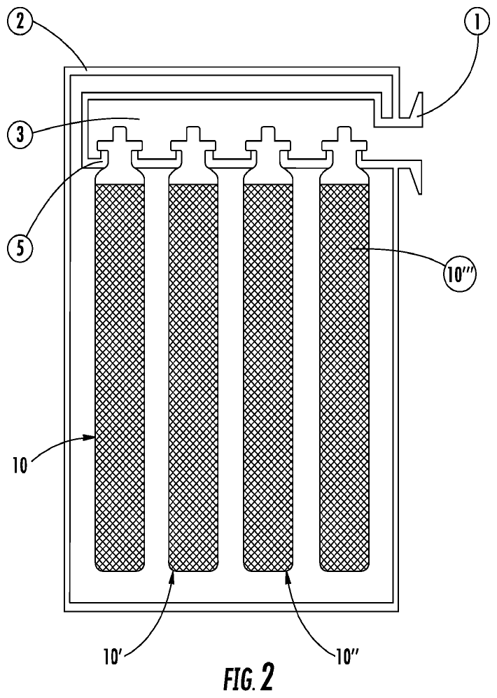

FIG. 2 shows an embodiment wherein a first container 2 surrounds a second container 5 comprising a plenum chamber 3 and a plurality of independent or discrete microcarrier receiving regions 10, 10′, 10″ and 10′″. In certain embodiments, each region 10, 10′, 10″ and 10′″ is a porous mesh filter bag. Bead-containing fluid passes through a fitment 1 and into the plenum 3, where it distributes to mesh bags which capture the beads as the suspensory fluid passes through the mesh and into the first container 2. In this embodiment, the inlet port 1 is located on a side wall of the container 2. The mesh bags have a porosity sufficient to allow process fluid to pass while retaining the microcarriers within the mesh bags. Suitable porosities for the microcarrier receiving regions include 50-100 μm meshes.

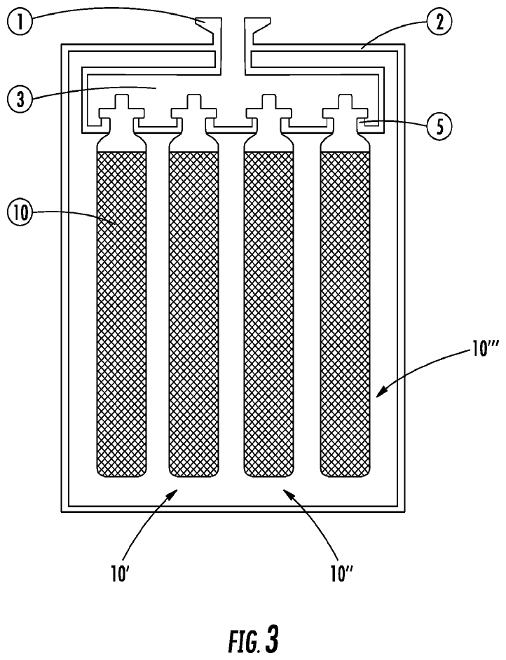

FIG. 3 shows an embodiment similar to FIG. 2, except that the fitment 1 providing access to the plenum 3 of the second container is located on the top of the apparatus. The fitment 1 can provide support for the apparatus if it engages a hook or slotted support, for example.

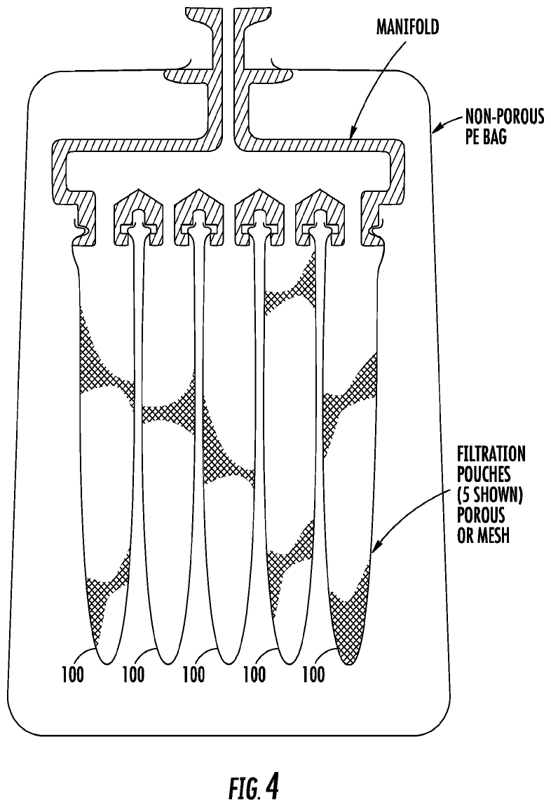

FIG. 4 illustrates an embodiment where the second container comprises a plurality of discrete filtration pouches 100. Each filtration pouch may be attached to a manifold and is in fluid communication with an inlet to the first container, such as a non-porous polyethylene bag. The attachment may be mechanical, or if both the second container (or the relevant portion thereof) and the manifold are the same material (e.g., PE), then they can be heat sealed.

In certain embodiments, each pouch 100 is a mesh pouch or other porous material, configured to contain a plurality of microcarriers while allowing fluid to pass through.

In some embodiments, the second container may be pre-loaded with microcarriers, and the apparatus may be used to wash the microcarriers with a process liquid, such as to wash adherent cells off of the microcarriers, or to adhere cells in the process liquid to the microcarriers.

A hypothetical microcarrier receiving region can be represented by the following example. A cube with dimensions 10×10×10 has sidewall surfaces of 10×10×5, since excluding the top wall there are five walls of 10×10 units=500 square units. If this is replaced by 10×1 unit pouches as microcarrier receiving regions, then the total side and bottom wall surfaces would be 10×10 (2 each large sidewalls×1 unit) plus 10×1×3 (2 short sidewalls plus 1 bottom wall for each pouch) or 2300 square units of filtration area, a 460% increase in approximately the same space.

In use, in certain embodiments the described filter device is attached to a port. The port in turn is attached by tubing to a pump or gravity flow circuit draining suspension from a cell culture vessel. That flow is directed to the microcarrier receiving regions such as filtration mesh. The access to the microcarrier receiving regions is either by direct attachment to the port or else through an extension tube from the port that accesses the first container (FIG. 2). The microcarrier solution passes into the upper part of the second compartment, which functions as a plenum, and the microcarrier solution is distributed to the microcarrier receiving regions, such as pouches, bags or pleated bags of mesh filter fabric or porous sheeting (FIG. 3). Because the additional surface area provided by the sidewalls of the microcarrier receiving regions exponentially multiplies the surface area for filtration as compared to a standard filter unit having only one microcarrier receiving region, the apparatus is also exponentially more efficient over the prior art filters.

Suitable microcarriers include CYTODEX microcarriers available from GE; SOLOHILL microcarriers available from Pall, and CELLBIND microcarriers available from Corning.

### Example

A filtration device has a first container such as a plastic or polyethylene bag, and a second container comprised of a plenum and five mesh filter bags wherein each filter bag has filter mesh fabric dimensions of 2 cm×10 cm×10 cm for a total area of 260 cm² per individual bag. 100 liters of Cytodex 3 microcarrier beads (141-211 micron diameter) in CHO cell culture fluid is pumped into the described bead filtration device which has a mesh size of 80 microns. The volume of swollen Cytodex 3 beads is 50 milliliters (ml) per liter of pumped bead solution, for a final packed bead volume of 500 mls/100 liters of bead suspension. The second container of the bead filter has five mesh bags attached to the plenum of the container. Five bags will capture 500 ml of beads when 100 liters of bead containing fluid is processed. It's not necessary for the bags to fill exactly evenly, however, they will tend to do this. If one bag is substantially fuller than another, then the fuller bag will have a slightly higher pressure drop, and incoming liquid will be biased towards the less full/lower pressure drop bags. At this point this leaves 600 cm² of as yet unobstructed filter media above the accumulated beads. This compares to a second container of the prior art, which is comprised of only one bag of the same outer dimensions, i.e., 10 cm×10 cm×10 cm, wherein the amount of unobstructed filter medium not covered by captured beads is only 200 cm². That is one third as much filtration area as that provided by the invention of claim 1 in this example. Thus the unobstructed flow rate of the claimed invention at one half exhaustion of the available filtration medium will be three times that of the prior art device in this example.

What is claimed is:

1. A container for separating microcarriers from a process fluid, comprising:
a first compartment;
an inlet port providing a fluid path into the first compartment;
an outlet port providing a fluid path exiting the first compartment; and
a second compartment disposed inside the first compartment and fluidly connected with the inlet port of the first compartment, said second compartment comprising a plurality of discrete microcarrier receiving regions.

2. The container of claim 1, wherein each microcarrier receiving region comprises porous mesh having a porosity sufficient to allow process fluid to pass while retaining said microcarriers.

3. The container of claim 1 wherein the microcarrier receiving regions in fluid communication with a plenum to form a manifold.

4. The container of claim 1, wherein each microcarrier receiving region comprises a mesh bag coupled to a manifold that is fluidly connected to an input port of the first compartment.

5. The container of claim 1, wherein each microcarrier receiving region comprises a porous pleated bag.
